(19) United States
(12) Patent Application Publication
(10) Pub. No.: US 2026/0144840 A1
(43) Pub. Date: May 28, 2026

(54) TOPICAL FORMULATION FOR BINDING TO DERMATOLOGICAL CANNABINOID RECEPTORS

(71) Applicant: Jessica KADO, West Bloomfield, MI (US)

(72) Inventor: Jessica KADO, West Bloomfield, MI (US)

(21) Appl. No.: 19/451,857

(22) Filed: Jan. 16, 2026

Related U.S. Application Data
(63) Continuation-in-part of application No. 17/710,079, filed on Mar. 31, 2022, now Pat. No. 12,527,829.
Application No. 17/710,079 is a division of application No. 16/750,153, filed on Jan. 23, 2020, now Pat. No. 11,318,179.
Application No. 16/750,153 is a continuation-in-part of application No. 15/627,745, filed on Jun. 20, 2017, now Pat. No. 10,543,176.

(51) Int. Cl. A61K 36/53; A61K 9/06; A61K 36/236; A61K 36/28; A61K 36/61; A61K 36/752

(52) U.S. Cl. CPC A61K 36/53; A61K 9/06; A61K 36/236; A61K 36/28; A61K 36/61; A61K 36/752

(57) ABSTRACT

A topical cream or ointment is provided that provides for the binding to CB1 and/or CB2 receptors for the soothing of pain, inflammation of smooth muscle, tendons, ligaments, skeletal muscle, endothelial cell, synovial cell, peripheral nerve fibers, reduction of endothelial bruising, promotion of healing of endothelial bruising, and dermatological itch sensation wherein the improvement comprises providing the effect of cannabinoid using plant extracts. A topically administered formulation is also provided for treating anal mucosal inflammation, soothing of pain, inflammation of anal mucosal inflammation; reducing keratinocyte apoptosis; treating viral-associated epithelial damage, treating radiation dermatitis; and treating chemotherapy-induced dermatitis.

### CROSS-REFERENCE TO RELATED APPLICATIONS

This application is a US Continuation-in-Part application of U.S. Divisional application Ser. No. 17/710,079, filed Mar. 31, 2022, which claims the priority from US Continuation-in-Part application Ser. No. 16/750,153, filed Jan. 23, 2020, no U.S. Pat. No. 11,318,179, which claims priority from U.S. Non-Provisional application Ser. No. 15/627,745, now U.S. Pat. No. 10,543,176, filed Jun. 20, 2017, the contents of each are incorporated herein by reference in their entirety for all purposes.

### FIELD

The present invention is relevant to the field of suppressing sensory afferent neuron symptoms of itch and, more particularly, to itch suppression through the binding of agents to dermatological cannabinoid receptors and compositions for the same.

### BACKGROUND

Most recently, research into the topical use of cannabinoids has found therapeutic dermatological effects of such compounds. Numerous studies have investigated medical uses of cannabinoids for chronic pain, spacticity, anorexia and nausea. Topical therapeutic uses have further been found for the treatment of dermatological conditions including pruritus, inflammatory skin diseases and skin cancer.

Such recent findings indicate cannabidiol (CBD) and cannabigerol (CBG) bind to Cannabinoid Receptor Type 1 (CB1) and/or Cannabinoid Receptor Type 2 (CB2) receptors, resulting in therapeutic effects in treating psoriasis, pruritus, allergic contact dermatitis, atopic dermatitis and other eczematous dermatoses. In light of such recent knowledge, persons having ordinary skill in the relevant art may use cannabinoids to stimulate or to bind to cannabinoid receptors in the skin in order to excite or prevent a response and, in particular, to prevent itching.

Receptors throughout the body that are part of the endocannabinoid system are known to be involved in a variety of physiological processes. However, while cannabinoids are currently allowed in twenty eight states for medical applications, currently exist some insurmountable barriers to reliance of synthetic or process products of medical marijuana in the treatment of dermatological itch. These include, inter alia, lack of availability in 22 states; heightened regulatory scrutiny and its impact on the supply chain; requirement o doctor prescription and supervision; non-medical ‘political’ negative reactions to use of a cannabis related product; and, most significantly, the costs associated with the inclusion of cannabinoid products into a topical formulation. Both the use of cannabis related ingredients and such cost are especially barriers in the creation of an over-the-counter topical agent.

Consequently, a need exists for the use of binding to CB1 and/or CB2 receptors in a topical ointment in the soothing of dermatological itching. A further need exists for such a topical ointment that emulates the use of cannabinoid without the actual use of cannabinoids.

### SUMMARY

Aspects of the invention are summarized below to aid in the understanding of embodiments of the invention and the application. Yet, the invention is fully defined by the claims of the applications.

It is thus an object of the present invention to provide a topical ointment for the treatment of dermatological itching through binding to the CB1 and/or CB2 receptors.

It is a further object of the present invention to provide for CB1 and/or CB2 receptor bindings without the use of cannabinoid ingredients.

It is a feature of the present invention to provide a topical cream or ointment that utilizes a blend of aromatics and essential oils in a proportion and amount that emulates the use of cannabinoids, and at a commercially effective cost.

The present invention provides a topical formulation for binding to dermatological cannabinoid receptors. Essential oil extracts are provided and blended in a proportion such as to create a phyto-pseudo-cannabinoid formulation. The phyto-pseudo-cannabis formulation comprises extracts of sweet almond, safflower, vitamin E, chamomile, Avocado, lavender, lemon balm, vitamin A, Vitamin D, corn, pansy (viola tricolor) clove, rosemary, yarrow, and ylang ylang. These leaf, flower and herb oils and extracts are provided in a proportion and in small amounts and with binders and stabilizers. Though such natural extracts can generally each be expensive to obtain, the use of the combination allows for such relatively small amounts to be effective and to allow for the finished topical cream or ointment to be inexpensive enough to allow for competitive commercial applications.

According to one approach, a topical cream or ointment is provided that provides for the binding to CB1 and/or CB2 receptors for the soothing of pain, inflammation of smooth muscle, tendons, ligaments, skeletal muscle, endothelial cell, synovial cell, peripheral nerve fibers, reduction of endothelial bruising, promotion of healing of endothelial bruising, and dermatological itch sensation wherein the improvement comprises providing the effect of cannabinoid using plant extracts.

According to one approach, a topical cream is provided for treating dermatologic itch, and soothing of pain and inflammation of smooth muscle, tendons, ligaments, skeletal muscle, endothelial cell, synovial cell, peripheral nerve fibers, reduction of endothelial bruising, promotion of healing of endothelial bruising, using a topical application, and may have: 0.70% wt of an Essential Oil Blend comprising a composition of Clove Oil, Lavender Oil, Rosemary Oil, Ylang Ylang Oil, and Lemon Oil; 0.1-1% wt Additional Lavender; 0.1-1% wt Additional Lemon Extract; 1.5% wt Pansy (Viola Tricolor) Extract; 0.1-1% wt Chamomile Flower Extract; >1% wt Ceramides; >1% wt Licorice Root; and >1% wt Yarrow.

According to one approach, a topical cream or ointment is provided that provides for the binding to CB1 and/or CB2 receptors for the soothing of smooth muscle, tendons, ligaments, skeletal muscle, endothelial cell, synovial cell, peripheral nerve fibers using plant extracts is provided using plant extracts. In one approach the topical cream or ointment may have essentially: Prunus amygdalus dulcis (Sweet Almond) Oil; Glycerin; Isopropyl Palmitate; Carthamus tinctorius (Safflower) Seed Oil; Stearic Acid; Tocopheryl (Vitamin E) Acetate; Glyceryl Stearate SE; Persea gratissima (Avocado) Oil; Chamomilla recutitia (Chamomile) Flower Extract; Lavandula angustifolia (Lavender) Flower Extract; Melissa Officinalis (Lemon Balm) Leaf Extract; Tocopherol (Vitamin E); Zea mays (Corn) Oil; Retinyl Palmitate (Vitamin A); Cholecalciferol (Vitamin D); Polysorbate 80; Cetyl Alcohol; Aminomethyl Propanol; Sorbitan Oleate; Carbomer; Tetrasodium Glutamate Diacetate; Pansy (Viola Tricolor) Extract; Polyacrylate-13; Polyisobutene; Polysorbate 20; Eugenia Caryophyllus (Clove) Flower Oil; Lavandin Grosso (Lavender) Oil; Rosmarinus officinalis (Rosemary) Leaf Oil; Canaga odorata (Ylang Ylang) Flower Oil; Melissa officinalis (Lemon Balm) Leaf Oil; Dipotassium Glycyrrhizinate; Achillea millefolium (Yarrow) Flower Extract; Ceramide 3; Ceramide 6 II; Ceramide 1; Phytosphingosine; Cholesterol; Sodium Lauroyl Lactylate; Carbomer; Xanthan Gum; and Phenoxyethanol Ethylhexyl Glycerin.

In another approach, a topically administered formulation is provided for treating anal mucosal inflammation, soothing of pain, inflammation of anal mucosal inflammation; reducing keratinocyte apoptosis; treating viral-associated epithelial damage, treating radiation dermatitis; treating chemotherapy-induced dermatitis; treating dermatologic itch; and soothing of pain and inflammation of smooth muscle, tendons, ligaments, skeletal muscle, endothelial cell, synovial cell, or peripheral nerve fibers, reduction of endothelial bruising, or promotion of healing of endothelial bruising using a topical application, wherein the formulation provides for the binding to CB1 and/or CB2 receptors for wherein the improvement comprises providing the effect of cannabinoid using plant extracts.

In this approach, the formulation of claim 11, may also have a topically administered effective amount of the formulation targeting treatment areas having: 0.70% wt formulation of an Essential Oil Blend comprising a composition of Clove Oil, Lavender Oil, Rosemary Oil, Ylang Ylang Oil, and Lemon Oil; 0.1-1% wt formulation Additional Lavender; 0.1-1% wt formulation Additional Lemon Extract; 1.5% wt Pansy (Viola Tricolor) Extract; 0.1-1% wt formulation Chamomile Flower Extract; >1% wt Ceramides; >1% wt Licorice Root; and >1% wt formulation Yarrow.

A method is provided for treating anal mucosal inflammation, soothing of pain, inflammation of anal mucosal inflammation; reducing keratinocyte apoptosis; treating viral-associated epithelial damage, treating radiation dermatitis; treating chemotherapy-induced dermatitis; treating dermatologic itch; and soothing of pain and inflammation of smooth muscle, tendons, ligaments, skeletal muscle, endothelial cell, synovial cell, or peripheral nerve fibers, reduction of endothelial bruising, or promotion of healing of endothelial bruising, comprising applying to the affected area the topical formulation.

Applying the topical formulation is provided to treat anal mucosal inflammation reduces pruritus, decreases venous congestion, and improves lymphatic drainage, wherein the topical formulation provides bacteriostatic protection to compromised anal mucosa, and wherein the essential oils comprise at least clove oil, lavender oil, rosemary oil, lemon balm oil, and ylang ylang oil and provides local anesthetic-like relief mediated by clove oil and chamomile.

This is effective in reducing inflammatory cytokine signaling associated with hemorrhoidal irritation.

A method is also provided for reducing keratinocyte apoptosis in UV-B-damaged skin, comprising topically applying a formulation comprising a phyto-pseudo-cannabinoid blend of essential oils, wherein the formulation decreases caspase-mediated apoptosis and reduces erythema, wherein the formulation comprises clove oil and Viola tricolor extract as apoptosis-modulating antioxidants, and wherein application results in decreased peeling and accelerated epidermal repair.

A method is also provided for treating viral-associated epithelial damage, comprising applying a topical formulation with and a phyto-pseudo-cannabinoid blend to an area infected with HSV or VZV, wherein the formulation down-regulates viral-triggered apoptotic signaling in keratinocytes; wherein the formulation reduces post-herpetic neuropathic irritation by modulating CB2 receptor signaling, and wherein the formulation comprises lemon balm extract, pansy extract, licorice root extract, and clove oil.

A method is also provided for treating radiation dermatitis, comprising applying to irradiated skin a topical formulation including a phyto-pseudo-cannabinoid blend, wherein the formulation decreases radiation-induced inflammation and promotes epithelial regeneration; wherein the formulation reduces keratinocyte apoptosis caused by ionizing radiation; and wherein the formulation prevents secondary infection and reduces treatment delays.

A method is also provided for treating chemotherapy-induced dermatitis, comprising applying to affected skin a topical formulation including comprising a phyto-pseudo-cannabinoid blend of plant extracts configured to reduce cytotoxic inflammation and accelerate skin healing, wherein the formulation restores the stratum corneum barrier through ceramide supplementation, and wherein antioxidant and anti-inflammatory activity minimizes collateral tissue injury caused by chemotherapeutic agents.

Further, the ingredients themselves at the levels provided are each generally considered safe and, as such, a finished topical cream or ointment should be viable for over-the-counter sales.

### DETAILED DESCRIPTION

It should be understood that the leg al scope of the description is defined by the words of the claims set forth at the end of this patent and that the detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

Cannabinoid receptors are of a class of cell membrane receptors under the G protein-coupled receptor superfamily. Cannabinoid receptors are known to be activated by three major groups of ligands: endocannabinoids, produced by the mammillary body; plant cannabinoids (such as Cannabidiol, produced by the cannabis plant); and synthetic cannabinoids (such as HU-210). All of the endocannabinoids and plant cannabinoids are lipophilic, such as fat soluble compounds.

In providing a topical medicament for the treatment of dermatological itch, a composition that provides for binding to CB1 and/or CB2 receptors have been found to be effective. It has been found that the use of a composition of plant extracts can mimic the endocannabinoids and plant cannabinoids binding to CB1 and/or CB2 receptors, and that such receptor occupancy can diminish pain, inflammation and dermatological itch sensation. According to one approach, the topical formulation for binding to smooth muscle, skeletal muscle, tendons, ligaments, endothelial cell, synovial cell, peripheral nerve fiber cannabinoid receptors to reduce pain, inflammation is provided. The current formulations also reduces endothelial bruising and promotes endothelial bruise healing.

According to one aspect of the present invention the topical preparation is provided as a topical cream or ointment that utilizes a blend of aromatics and essential oils in a proportion and amount that emulates this CB1 and/or CB2 receptor binding. Essential oil extracts are provided and blended in a proportion such as to create a phyto-pseudo-cannabinoid formulation. Most suitably the formulation contains the following:

| % wt | Ingredient |
|---|---|
| 0.70 | Essential Oil Blend (comprised of Clove Oil, Lavender Oil, |
| | Rosemary Oil, Ylang Ylang Oil, and Lemon Oil) |
| 0-1 | Additional Lavender |
| 0-1 | Additional Lemon Extract |
| 1.5 | Pansy (Viola Tricolor) Extract |
| 0-1 | Chamomile Flower Extract |
| >1 | Ceramides |
| >1 | Licorice Root |
| >1 | Yarrow |

Caryophyllene is a sesquiterpene found in high amounts of several Essential Oils, including Black Pepper, Ylang Ylang, and Melissa essential oils. It activates a CB2 receptors with psychoactive activity. The essential oil blend that includes the five listed essential oils all include some level of caryophyllene to bind to the CB2 receptor and help reduce an urge to itch your skin.

According to another aspect of the present invention topical cream or ointment formulation may contain the following: sweet almond; safflower; vitamin E; chamomile; Avocado; lavender; lemon balm; vitamin A; Vitamin D; corn; pansy (viola tricolor) clove; rosemary; yarrow; and ylang ylang. These leaf, flower and herb oils and extracts are provided in a proportion and in small amounts and with binders and stabilizers. Though such natural extracts can generally each be expensive to obtain, the use of the combination allows for such relatively small amounts to be used overall such as to allow for the finished topical cream or ointment to be inexpensive enough to allow for competitive commercial applications.

According to yet another aspect of the present invention, including binders and stabilizers, the topical cream or ointment formulation may contain the following: Water, Prunus amygdalus dulcis (Sweet Almond) Oil, Glycerin, Isopropyl Palmitate, Carthamus tinctorius (Safflower) Seed Oil, Stearic Acid, Tocopheryl (Vitamin E) Acetate, Glyceryl Stearate SE, Persea gratissima (Avocado) Oil, Chamomilla recutitia (Chamomile) Flower Extract, Lavandula angustifolia (Lavender) Flower Extract, Melissa officinalis (Lemon Balm) Leaf Extract, Tocopherol (Vitamin E), Zea mays (Corn) Oil, Retinyl Palmitate (Vitamin A), Cholecalciferol (Vitamin D), Polysorbate 80, Cetyl Alcohol, Aminomethyl Propanol, Sorbitan Oleate, Carbomer, Tetrasodium Glutamate Diacetate, Pansy (Viola Tricolor) Extract, Polyacrylate-13, Polyisobutene, Polysorbate 20, Eugenia Caryophyllus (Clove) Flower Oil, Lavandin Grosso (Lavender) Oil, Rosmarinus officinalis (Rosemary) Leaf Oil, Canaga odorata (Ylang Ylang) Flower Oil, Melissa officinalis (Lemon Balm) Leaf Oil, Dipotassium Glycyrrhizinate, Achillea millefolium (Yarrow) Flower Extract, Ceramide 3, Ceramide 6 II, Ceramide 1, Phytosphingosine, Cholesterol, Sodium Lauroyl Lactylate, Carbomer, Xanthan Gum, Phenoxyethanol Ethylhexyl Glycerin.

According to still a final aspect of the present invention, the topical cream or ointment formulation may contain the following:

| % wt | Ingredient |
|---|---|
| | Water |
| | Prunus Amygdalus Dulcis (Sweet Almond) Oil |
| | Glycerin |
| | Isopropyl Palmitate |
| | Carthamus Tinctorius (Safflower) Seed Oil |
| | Stearic Acid |
| | Tocopheryl (Vitamin E) Acetate |
| | Glyceryl Stearate SE |
| | Persea Gratissima (Avocado) Oil |
| | Chamomilla Recutitia (Chamomile) Flower Extract |
| | Lavandula Angustifolia (Lavender) Flower Extract |
| | Melissa Officinalis (Lemon Balm) Leaf Extract |
| | Tocopherol (Vitamin E) |
| | Zea Mays (Corn) Oil |
| | Retinyl Palmitate (Vitamin A) |
| | Cholecalciferol (Vitamin D) |
| | Polysorbate 80 |
| | Cetyl Alcohol |
| | Aminomethyl Propanol |
| | Sorbitan Oleate |
| | Carbomer |
| | Tetrasodium Glutamate Diacetate |
| | Pansy (Viola Tricolor) Extract |
| | Polyacrylate-13 |
| | Polyisobutene |
| | Polysorbate 20 |
| | Eugenia Caryophyllus (Clove) Flower Oil |
| | Lavandin Grosso (Lavender) Oil |
| | Rosmarinus Officinalis (Rosemary) Leaf Oil |
| | Canaga Odorata (Ylang Ylang) Flower Oil |
| | Melissa Officinalis (Lemon Balm) Leaf Oil |
| | Dipotassium Glycyrrhizinate |
| | Achillea Millefolium (Yarrow) Flower Extract |
| | Ceramide 3 |
| | Ceramide 6 II |
| | Ceramide 1 |
| | Phytosphingosine |
| | Cholesterol |
| | Sodium Lauroyl Lactylate |
| | Carbomer |
| | Xanthan Gum |
| | Phenoxyethanol Ethylhexyl Glycerin. |

These ingredients composition of the present inventions provides a new and surprising synergistic effect.

### 2. Operation of the Preferred Embodiment

In operation, a topical cream or ointment is provided that provides for the binding to CB1 and/or CB2 receptors for the soothing of dermatological itching. The cream or ointment composition provides the effect of cannabinoid without the actual use of cannabinoids. Several essential oils, including Black Pepper, Ylang Ylang, and Melissa essential oils in combination, contain caryophyllene, a sesquiterpene, in effective amounts to bind to the CB2 receptor and help reduce an urge to itch the skin.

Additional Embodiments may include:
1. Mucosal application to anus for decreased inflammatory signaling to anal hemorrhoids. Topical application leads to less pruritus, comforting anesthesia, decreased venous congestion and improved lymphatic flow leading to faster resolution and immediate comfort. The bacteriostatic properties of the formula also protect against secondary infections when the mucosal lining is injured or compromised during acute flares. Also same claims but in mucosa instead of skin.
2. Decrease in apoptosis cells and cell death in uv b mediated sunburned skin with anesthesia relief. Same formula Or can say most likely related to clove and viola tricolor specifically.
3. Antiviral and Down-regulates keratinocyte apoptotic death signaling related to viral infections (specifically hsv and vzv) and neuropathy related to those infections as well.

Other embodiments include using the creams for radiation dermatitis and chemo therapeutic dermatitis. Radiation dermatitis induces an ionizing radiation cytotoxic cellular response and then the chemotherapy induces a chemical cytotoxic cellular response. So the present embodiments work through the antioxidant pathway and then also enhances repair mechanisms so that people can recover not only more quickly but then also minimize the collateral damage within normal skin which is usually the skin that they have to bypass through for radiation, or the skin that gets the diffusion of the chemotherapy so it deposits in the skin or has to pass through the skin and the skin is just collateral damage. A lot of the times patients will get infections or secondary changes which will inhibit additional therapy. This is sometimes a limiting step for treatment for certain people because they can't get additional therapy until the skin heals or where they get an infection and that has to be taken care of before they can proceed with therapy. So, the idea is this will rebalance the skin profile inflammation cascades so that they can better be optimized with less collateral damage but then also more significantly quicker healing. The components of the embodiments are generally recognized as safe ingredients and there is now clinical data to show this.

In these embodiments, the topical phyto-pseudo-cannabinoid formulation is applied to mucosal tissues, including tissues that contain transitional epithelium, mucosal glands, and highly vascularized support stroma. In one particular approach, the formulation is applied to the anal mucosa, which differs structurally and immunologically from keratinized epidermis and demonstrates heightened sensitivity to inflammatory cytokine release during hemorrhoidal flares. When applied to the anus or perianal mucosa, the formulation binds to mucosal CB1 and CB2 receptors, resulting in reduced nociceptive firing, decreased afferent pruritus signaling, and decreased neurogenic inflammation. Essential oils contained within the formulation—including clove, yarrow, pansy, lavender, and lemon balm—provide both local anesthetic-like effects and anti-microbial, bacteriostatic, and soothing actions, thereby decreasing venous congestion, improving lymphatic drainage, and protecting against secondary mucosal infections. These effects collectively accelerate resolution of hemorrhoidal irritation and provide immediate comfort while supporting mucosal barrier restoration. The same mechanisms described for dermatologic tissues apply to mucosal tissues, with the added benefit of mucosal receptor density and enhanced local drug penetration.

In still further embodiments, the formulation decreases UV-B-mediated apoptosis in sunburned skin. Ultraviolet radiation induces DNA photoproducts, oxidative stress, and activation of apoptotic pathways in keratinocytes, resulting in erythema, pain, and subsequent peeling. Components of the formulation-particularly clove oil (rich in eugenol) and Viola tricolor (pansy) extract, as well as chamomile and rosemary-provide potent antioxidant scavenging activity and inhibition of caspase-mediated apoptotic pathways. Application of the formulation to sunburned skin limits keratinocyte apoptotic death, reduces inflammatory mediators such as TNF-α and IL-1β, and provides cooling anesthesia and pain reduction, thus accelerating epidermal healing.

In yet another embodiment, the formulation demonstrates antiviral modulatory activity and down-regulation of keratinocyte apoptosis associated with viral infection, particularly in conditions such as herpes simplex virus (HSV-1, HSV-2) infection and varicella-zoster virus (VZV) reactivation. Viral infection induces mitochondrial stress, oxidative injury, and apoptotic signaling in keratinocytes and epithelial cells, contributing to vesicle formation, neuropathic pain, and delayed skin recovery. Plant extracts such as clove, lemon balm (known for antiviral properties), pansy, and licorice root help attenuate viral-triggered apoptotic cascades, reduce inflammation along sensory nerve endings, and mitigate post-herpetic neuropathic irritation. The cannabinoid-mimicking receptor binding further decreases nociceptive conduction and supports recovery of virally stressed tissues.

In further embodiments, the topical formulation is used to treat radiation dermatitis, which occurs secondary to ionizing radiation exposure during cancer therapy. Ionizing radiation produces direct DNA strand breaks and indirect oxidative injury, triggering keratinocyte apoptosis, microvascular damage, and severe inflammation. Application of the formulation supports antioxidant activity, down-regulates inflammatory cytokine cascades, and promotes epithelial regeneration, thereby decreasing severity of erythema, desquamation, and ulceration. The formulation is useful for both acute radiodermatitis and chronic radiation-induced skin changes.

Additional embodiments include treatment of chemotherapy-induced dermatitis, in which cytotoxic agents cause inflammation, barrier disruption, and apoptotic cell death in normal skin as drug metabolites diffuse into dermal and epidermal tissues. By providing potent antioxidant activity, receptor-mediated anti-inflammatory signaling, and enhanced barrier repair through ceramides and botanical extracts, the formulation supports faster recovery and minimizes “collateral skin damage.” This allows patients to better tolerate cancer therapy cycles by reducing infections, preventing treatment delays, and maintaining skin integrity. All ingredients used in the formulation are generally recognized as safe (GRAS), enabling safe use even on compromised or sensitized skin.

The foregoing descriptions of specific embodiments of the present invention are presented for purposes of illustration and description. The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way. Any unintended embracement of such subject matter is hereby disclaimed. They are not intended to be exhaustive nor to limit the invention to precise forms disclosed and, obviously, many modifications and variations are possible in light of the above teaching. The embodiments are chosen and described in order to best explain principles of the invention and its practical application, to thereby enable others skilled in the art to best utilize the invention and its various embodiments with various modifications as are suited to the particular use contemplated. It is intended that a scope of the invention be defined broadly by the Drawings and Specification appended hereto and to their equivalents. Therefore, the scope of the invention is in no way to be limited only by any adverse inference under the rulings of Warner-Jenkinson Company, v. Hilton Davis Chemical, 520 US 17 (1997) or Festa Corp. v. Shoketsu Kinzoku Kogyo Kabushiki Co., 535 U.S. 722 (2002), or other similar caselaw or subsequent precedent should not be made if any future claims are added or amended subsequent to this Provisional Patent Application.

What is claimed is:

1.-10. (canceled)

11. A topically administered formulation for treating anal mucosal inflammation, soothing of pain, inflammation of anal mucosal inflammation; reducing keratinocyte apoptosis; treating viral-associated epithelial damage, treating radiation dermatitis; treating chemotherapy-induced dermatitis; treating dermatologic itch; and soothing of pain and inflammation of smooth muscle, tendons, ligaments, skeletal muscle, endothelial cell, synovial cell, or peripheral nerve fibers, reduction of endothelial bruising, or promotion of healing of endothelial bruising using a topical application, wherein the formulation provides for the binding to CB1 and/or CB2 receptors for wherein the improvement comprises providing the effect of cannabinoid using plant extracts.

12. The formulation of claim 11, further comprising a topically administered effective amount of the formulation targeting treatment areas having:
0.70% wt formulation of an Essential Oil Blend comprising a composition of Clove Oil, Lavender Oil, Rosemary Oil, Ylang Ylang Oil, and Lemon Oil;
0.1-1% wt formulation Additional Lavender;
0.1-1% wt formulation Additional Lemon Extract; 1.5% wt Pansy (Viola Tricolor) Extract;
0.1-1% wt formulation Chamomile Flower Extract; >1% wt Ceramides; >1% wt Licorice Root; and
>1% wt formulation Yarrow.

13. A method of treating anal mucosal inflammation, soothing of pain, inflammation of anal mucosal inflammation; reducing keratinocyte apoptosis; treating viral-associated epithelial damage, treating radiation dermatitis; treating chemotherapy-induced dermatitis; treating dermatologic itch; and soothing of pain and inflammation of smooth muscle, tendons, ligaments, skeletal muscle, endothelial cell, synovial cell, or peripheral nerve fibers, reduction of endothelial bruising, or promotion of healing of endothelial bruising, comprising applying to the affected area the topical formulation of claim 11.

14. The method of claim 13, wherein applying the topical formulation of claim 11 is used to treat anal mucosal inflammation reduces pruritus, decreases venous congestion, and improves lymphatic drainage, wherein the topical formulation provides bacteriostatic protection to compromised anal mucosa, and wherein the essential oils comprise at least clove oil, lavender oil, rosemary oil, lemon balm oil, and ylang ylang oil and provides local anesthetic-like relief mediated by clove oil and chamomile.

15. The method of claim 14, further comprising reducing inflammatory cytokine signaling associated with hemorrhoidal irritation.

16. A method of reducing keratinocyte apoptosis in UV-B-damaged skin, comprising topically applying a formulation of claim 11 also comprising a phyto-pseudo-cannabinoid blend of essential oils, wherein the formulation decreases caspase-mediated apoptosis and reduces erythema, wherein the formulation comprises clove oil and Viola tricolor extract as apoptosis-modulating antioxidants, and wherein application results in decreased peeling and accelerated epidermal repair.

17. A method of treating viral-associated epithelial damage, comprising applying a topical formulation of claim 11 and a phyto-pseudo-cannabinoid blend to an area infected with HSV or VZV, wherein the formulation down-regulates viral-triggered apoptotic signaling in keratinocytes; wherein the formulation reduces post-herpetic neuropathic irritation by modulating CB2 receptor signaling, and wherein the formulation comprises lemon balm extract, pansy extract, licorice root extract, and clove oil.

18. A method of treating radiation dermatitis, comprising applying to irradiated skin a topical formulation of claim 11 including a phyto-pseudo-cannabinoid blend, wherein the formulation decreases radiation-induced inflammation and promotes epithelial regeneration; wherein the formulation reduces keratinocyte apoptosis caused by ionizing radiation; and wherein the formulation prevents secondary infection and reduces treatment delays.

18. A method of treating chemotherapy-induced dermatitis, comprising applying to affected skin a topical formulation of claim 11 including comprising a phyto-pseudo-cannabinoid blend of plant extracts configured to reduce cytotoxic inflammation and accelerate skin healing, wherein the formulation restores the stratum corneum barrier through ceramide supplementation, and wherein antioxidant and anti-inflammatory activity minimizes collateral tissue injury caused by chemotherapeutic agents.
